(19) United States
(12) Patent Application Publication
(10) Pub. No.: US 2009/0215150 A1
(43) Pub. Date: Aug. 27, 2009

(54) Sample container and filtration apparatus and method of filtration using the same

(76) Inventors: Jeffrey Kane, Hudson, MA (US); Thomas Taylor, Windham, MA (US); Christopher M. Catinella, Worcester, MA (US); Ryan Neil Peter Hall, Marlborough, MA (US)

Correspondence Address: CANTOR COLBURN, LLP, 20 Church Street, 22nd Floor, Hartford, CT, 06103, US

(21) Appl. No.: 12/070,974

(22) Filed: Feb. 22, 2008

(51) Int. Cl. C12N 1/00; B01D 29/00

(52) U.S. Cl. 435/243; 422/101

(57) ABSTRACT

A sample container and filtration apparatus is described comprising a sealable sample container and a surface that is configured to interface with a vacuum base. When the surface interfaces with the vacuum base, a portion of the sample container is broached, providing a pathway for the vacuum source to pull the sample therethrough. In exemplary embodiments, the broachable portion of the sample container comprises an actuating valve. The method for using the sample container and filtration apparatus significantly reduces the possibility of contamination of the sample and during the sample testing procedure relative to the conventional devices and procedures, provides more consistent and reliable test results, and significantly reduces the number of preparation and testing steps for the testing procedure.

### BACKGROUND

A traditional form of sample testing, in particular water testing, utilizes a vacuum filtration system to pull the sample through a membrane for further culturing and analysis. The system uses a sample container for collecting and transporting the sample to a testing site and a vacuum base that is configured to attach to a vacuum manifold source. With reference to Prior Art FIGS. 1-2, a filter membrane is removed from sterile packaging (e.g., by opening the packaging and grabbing the membrane with metal forceps that have been flamed) and is placed on the vacuum base. A vacuum funnel is clamped thereover to secure the assembly prior to vacuum filtration of the sample.

In ideal practice, the funnel and the base are autoclaved overnight prior to use (Prior Art FIG. 3). A sample bottle is taken from storage and transported to a site of interest. Some amount of sample (e.g., 250 millileters (ml) of water) is placed in the sample container. The sample container is labeled and placed in a cooler for transport to the lab, and the sample is placed in a refrigerator at the lab until testing (Prior Art FIG. 4).

When the test environment is ready, the sample container is removed from the refrigerator (Prior Art FIG. 5). The sample container is opened, and a specified amount of sample (e.g., 100 ml) is poured into the funnel of the assembly (see Prior Art FIG. 6). The vacuum source is turned on via the manifold, and within 10 to 30 seconds, the filtrate is pulled through the membrane (Prior Art FIG. 7).

After vacuum filtration, the vacuum source is turned off, the funnel and the clamp are removed and placed aside (Prior Art FIG. 8). The forceps are flamed and extinguished. The filter is removed from the base with the forceps and placed in a Petri dish (Prior Art FIG. 9).

Subsequent to removing the filter, the filter and base are rinsed with distilled or filtered water (Prior Art FIG. 10), the funnel and base assembly are clamped together and are put in a Ultraviolet (UV) chamber for a period of time (e.g., five minutes) (Prior Art FIG. 11). The assembly is placed back on the manifold (Prior Art FIG. 12), the clamp and funnel are removed, and the base is flamed for a period of time (e.g., 10 to 15 seconds) (Prior Art FIG. 13). The interior of the funnel is also flamed for a period of time (e.g., 10 to 15 seconds) (Prior Art FIG. 14), and the assembly is clamped back together for the next sample testing (Prior Art FIG. 15).

As was noted above, the above steps are the recommended, ideal steps for ensuring that sample contamination is at a minimum. However, in practice, not all of the steps are typically performed, and sample contamination remains a problem with regard to the majority of the above iterated steps. The prior art would greatly benefit from solutions that would reduce or eliminate sample contamination with regard to one or more of the above described sample testing steps.

### SUMMARY

The above described and other disadvantages of the prior art are overcome and alleviated by the presently described sample container and filtration apparatus, which comprises a sealable sample container and a surface that is configured to interface with a vacuum base. When the surface interfaces with the vacuum base, a portion of the sample container is broached, providing a pathway for the vacuum source to pull the sample therethrough.

In an exemplary embodiment, the broachable portion of the sample container comprises an actuating valve. When the sample container is placed onto the base, the valve opens due to interaction between the valve and the base, and a pathway opens between the sample and the base.

In exemplary embodiments, a lower portion of the valve contacts an upper portion of the base during the mating of the sample container and the base. The valve is pressed upwardly into the sample container, and at least one pathway is opened thereby.

In other exemplary embodiments, a plurality of pathways are opened around the valve to more evenly distribute the sample across a filter, which is situated between the valve and the vacuum source.

In other exemplary embodiments, the interface provides an audible signal and/or provides a ridge or projection in groove interface for positive engagement between the sample container and the base.

The method for using the sample container and filtration apparatus significantly reduces the possibility of contamination of the sample and during the sample testing procedure relative to the conventional devices and procedures, provides more consistent and reliable test results, and significantly reduces the number of preparation and testing steps for the testing procedure.

### BRIEF DESCRIPTION OF THE DRAWINGS

Referring now to the drawings, wherein like elements are numbered alike in the following FIGURES:

PRIOR ART FIG. 1 illustrates a conventional installation of a filter membrane into a vacuum filtration assembly;

PRIOR ART FIG. 2 illustrates a conventional vacuum filtration assembly that is ready for use;

PRIOR ART FIG. 3 illustrates the ideal autoclaving of the conventional vacuum funnel and base prior to use;

PRIOR ART FIG. 4 illustrates a conventional sample container in storage prior to testing;

PRIOR ART FIG. 5 illustrates conventional transfer of a sample container from storage to the testing asembly;

PRIOR ART FIG. 6 illustrates conventional transfer of the sample from the sample container into the vacuum funnel;

PRIOR ART FIG. 7 illustrates conventional vacuum filtration of the sample;

PRIOR ART FIG. 8 illustrates removal of the conventional clamp and vacuum funnel;

PRIOR ART FIG. 9 illustrates transfer of the filter from the conventional base into a Petri dish;

PRIOR ART FIG. 10 illustrates conventional rinsing of the funnel and base;

PRIOR ART FIG. 11 illustrates conventional UV treatment of the vacuum filtration assembly;

PRIOR ART FIG. 12 illustrates conventional re-installation of the vacuum filtration assembly onto the vacuum manifold;

PRIOR ART FIG. 13 illustrates conventional removal of the vacuum funnel and flaming of the base;

PRIOR ART FIG. 14 illustrates conventional flaming of the vacuum funnel;

PRIOR ART FIG. 15 illustrates the start of a second conventional sample testing with the vacuum filtration assembly;

FIG. 16 is a perspective view of an exemplary sample container and filtration apparatus;

FIG. 17 is a cross sectional view of an exemplary sample container and filtration apparatus mated with an exemplary base;

FIG. 18 is a partial closup view of the mating of the exemplary sample container and filtration apparatus and the exemplary base; and

FIG. 19 is a perspective view of an exemplary sample container and filtration apparatus in an uninstalled state.

### DETAILED DESCRIPTION

Reference will now be made in detail to exemplary embodiments, examples of which are illustrated by the accompanying drawings. As indicated above, the presently described sample container and filtration apparatus includes a broachable surface during the interface of the sample container and a base that is configured to be connected to a vacuum source.

As will be understood from the above and from the following detailed description, the presently described sample container and filtration apparatus significantly reduces the possibility of contamination of the sample and during the sample testing procedure relative to the conventional devices and procedures. The presently described apparatus provides more consistent and reliable test results. The presently described apparatus also significantly reduces the number of preparation and testing steps for the testing procedure.

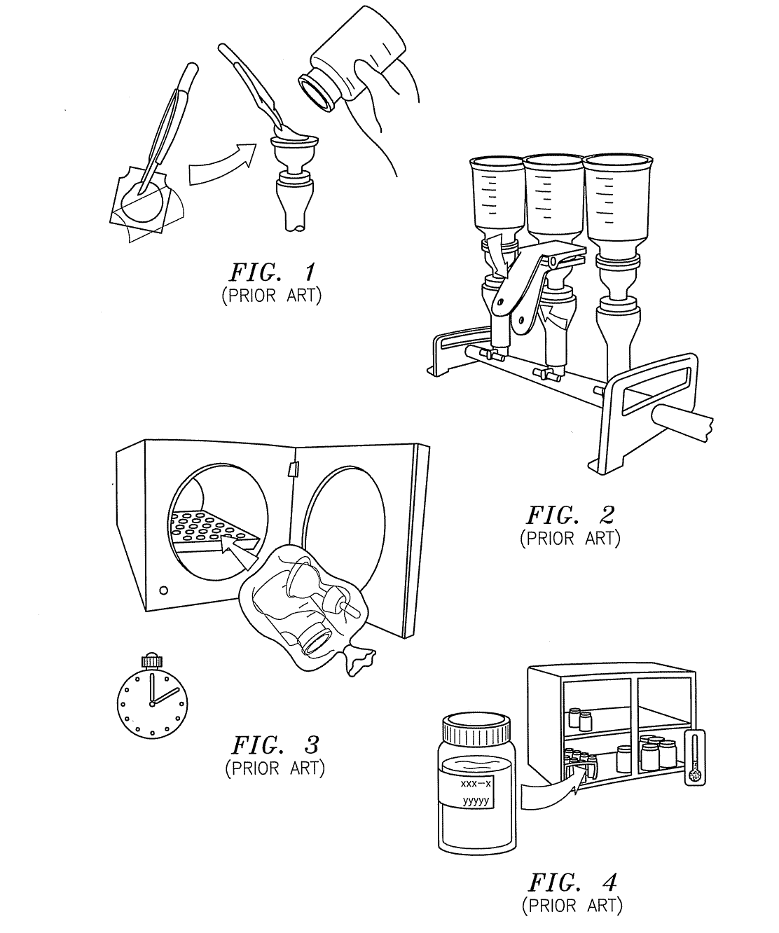

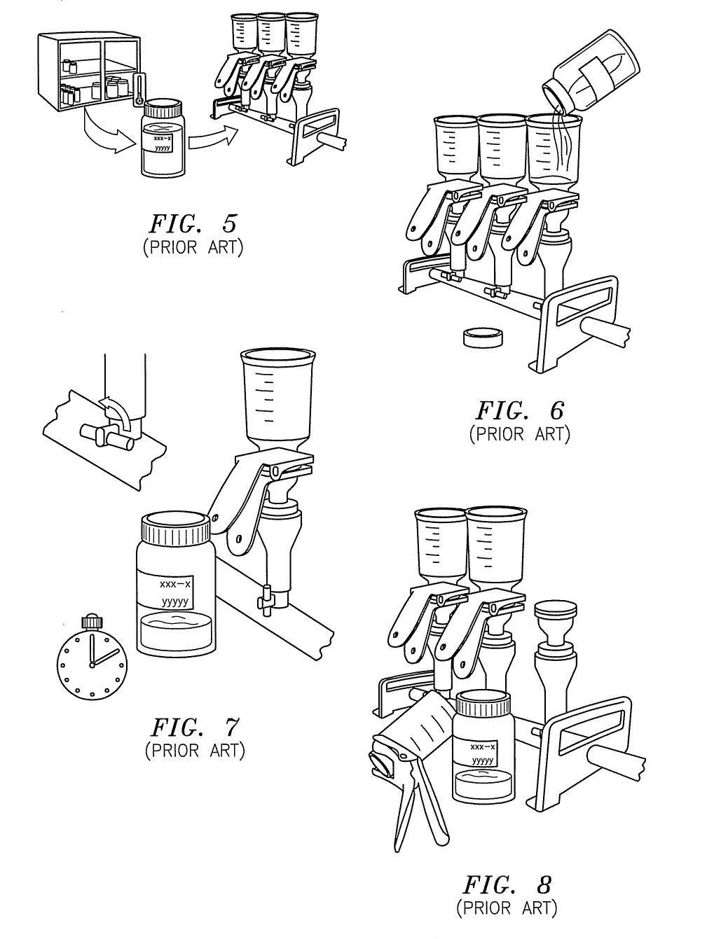

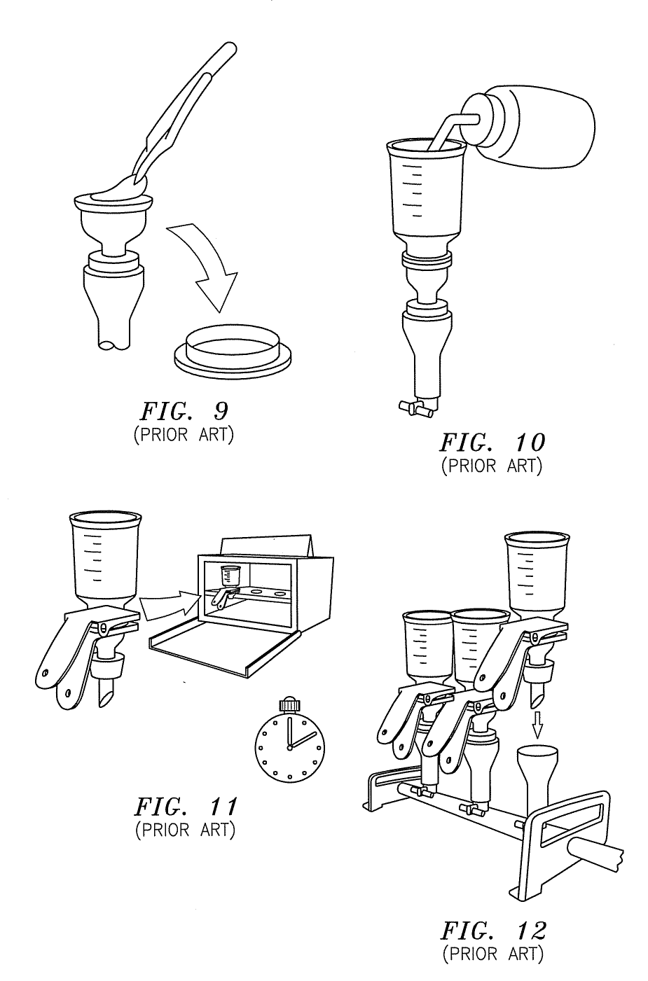

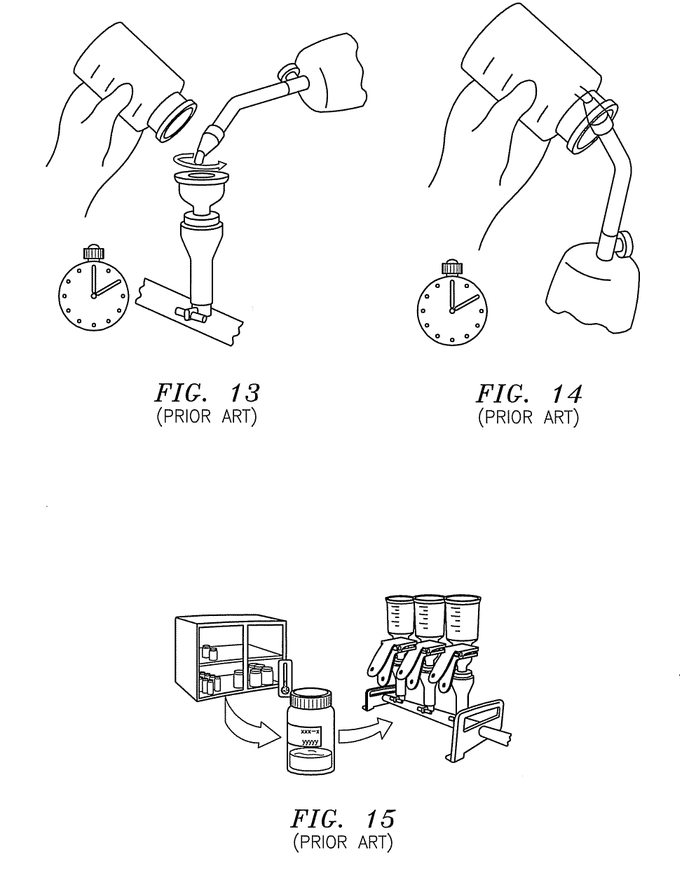

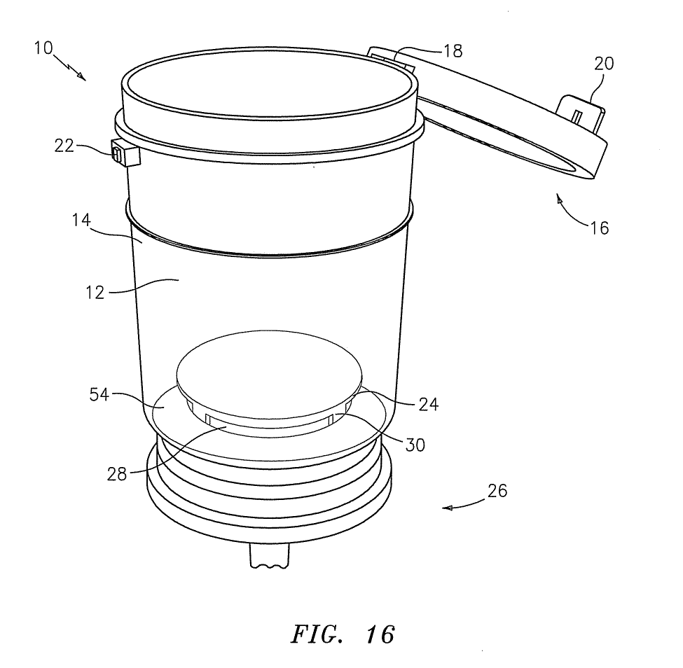

Referring now to FIG. 16, an exemplary sample container and filtration apparatus (hereinafter referred to simply as “sample container”) is illustrated generally at 10. The sample container includes an interior volume 12 configured to receive a predetermined amount of sample (e.g., 100 ml, 200 ml, or 250 ml, among others). In exemplary embodiments, a recommended level of sample is indicated by a fill line 14.

The exemplary sample container also includes a cover 16, illustrated in this exemplary embodiment as a cap with a living hinge 18 (although other embodiments are contemplated herein, including without limitation, threaded covers, etc.). In exemplary embodiments, the sample container is transported to a sample site, removed from a sterile container (e.g., sterile flexible packaging), and a sample amount is placed within the interior volume 14. The cover 16 is then closed to seal the sample therein. The cover 16 may interface with a complementary container surface in such a way to reduce the possibility of accidental re-opening, e.g., with a friction fit configuration, with a ridge or projection in groove configuration, with a locking configuration, etc.

In exemplary embodiments, the cover includes a locking member 20 that positively engages a projection 22 on the sample container. This tab may be pliable to permit re-opening of the cover, or the tab may be configured to break in order to re-open the cover. A breakable tab 20 provides benefit in serving as a positive indication that the sample has not been exposed to possible sources of contamination between the sample site and the testing site. During testing and just prior to applying vacuum, the tab may be broken to allow the cap to be opened such that a vacuum may be better drawn through the sample container. In the alternative, one or more vents (not shown) may be provided in the cover or on another surface of the sample container to better facilitate application of the vacuum.

The sample container 10 also includes a broachable portion 24. When the sample container is interfaced with a base that is configured to interface with a vacuum source, shown generally at 26, the broachable portion 24 exposes a pathway 28 between the interior volume 14 and the vacuum source.

In the illustrated exemplary embodiment, the broachable surface 24 comprises a sliding valve that is actuated into the interior volume 14 of the sample container 10 during mating of the sample container and the base. That is, as the sample container 10 is pressed over the base, an interface portion 30 of the sliding valve contacts the base 26 and forces the sliding valve to move into the interior volume exposing the pathway 28. While the following describes, as an exemplary embodiment, a sliding valve having vents, it should be recognized that the present invention is not limited thereto, but instead contemplates other forms of broachable surfaces as well. Thus, the following (with regard to the sliding valve, the vents or otherwise) should be read as being exemplary rather than limiting.

In exemplary embodiments, a plurality of pathways 28 is exposed. In the illustrated exemplary embodiment, the plurality of pathways 28 present as vents that evenly spaced around the circumferential periphery of the valve. Providing vents around portions of the circumferential periphery of the valve allows sample to be more evenly distributed over a filter that is provided between the valve and the vacuum source.

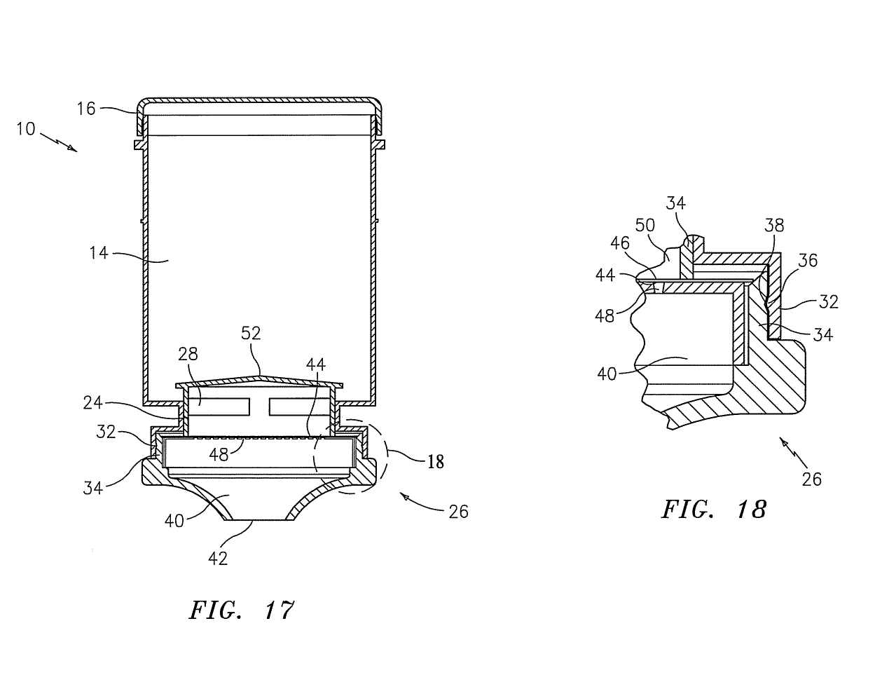

Referring now to FIG. 17, an exemplary embodiment is illustrated that elaborates on an interface between the sample container and the base. As before, the illustrated exemplary embodiment includes a cover 16, an interior volume 14 and a sliding valve 24. With additional reference to FIG. 18, the illustrated exemplary sample container 16 includes an interface surface 32 that is complementary to a base interface surface 34. This interface may be a friction fit, a stacking fit with supplemental mechanisms for preventing accidental disassociation of the interface (e.g., via magnets provided in or near the interface surfaces 32, 34), or, as illustrated, an interface that includes a ridge 36 in groove 38 (or the like, such as a projection in groove or projection in hole) configuration. In the case of the ridge in groove configuration or the like, the interface may be configured to generate an audible and/or tactile feedback indicative of positive mating.

Referring still to FIGS. 17 and 18, the illustrated exemplary base element comprises an interior volume 40, a vacuum source pathway 42, and a filter support surface 44, which provides support for a filter 46 and exposes the filter to the vacuum source. In the illustrated embodiment, the filter support surface includes a plurality of perforations 48.

The exemplary valve is shown in the installed state, with vents 28 exposed to the interior volume 14 of the sample container 10 through interaction with the base 26 during mating of the sample container and the base. In the installed state, an applied vacuum will draw the sample from the interior volume 14 of the sample container, through the vents 28 into an interior volume 50 of the valve 24, through the filter 44 and into the interior volume 40 of the base 26.

With reference to FIG. 17, the valve is illustrated with a sloped surface portion 52 (in the specific illustration, a conical surface). With reference to FIG. 16, the sample container is illustrated with sloping wall surfaces 54. These exemplary embodiments reduce or eliminate standing sample material at the end of the vacuum filtration process.

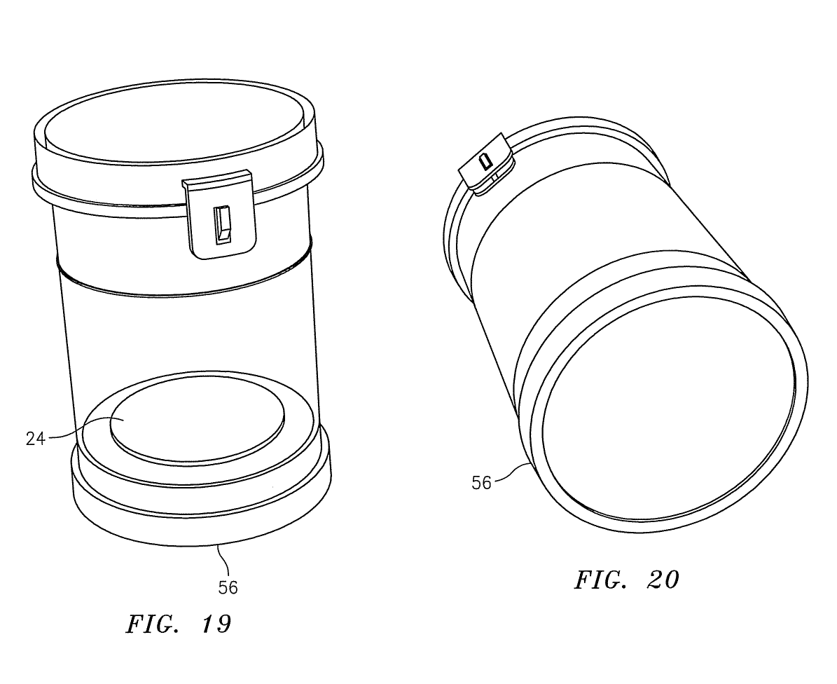

With reference to FIGS. 18 and 19, the sample container may also include a cover 56 provided over the broachable surface 24 to preserve a sterile field prior to testing and/or to prevent accidental broaching of surface 24. It is also noted that FIG. 18 illustrates the broachable surface 24 in an uninstalled state, wherein there is no pathway between the interior volume 14 of the sample container 10 and the exterior of the container.

In exemplary embodiments, the filter may be packaged with the sample container (i.e., between a cover 56 and the broachable surface 24. Alternatively, the filter may be packaged with the base, wherein the filter and the base are maintained in a sterile field (for example, flexible sterile packaging) prior to use. In other embodiments, the filter is separately maintained.

In exemplary embodiments, one or both of the sample container and the base are disposable products. By utilizing the presently described sample container and/or base as disposables, benefit is derived by virtue of the fact that the containers do not need to be cleaned and sterilized between testings. Rather, in the case of the sample container, the sample may simply be collected and tested, without autoclave, rinsing, flaming of surfaces, etc., and then may simply be thrown away in favor of another disposable sample container.

In exemplary embodiments, testing of a sample may easily be done by removing the sample container from a sterile field at a sample site, placing sample within the container, sealing the container, and transporting the container directly to the testing site (preferably in a cold environment). The sample container may then be placed on a base, with a filter material between the sample container and the base, a cap or other vent may be opened, and a vacuum source applied through the base. After vacuum filtration is finished, the filter is placed in a culture tray, and one or both of the sample container and base may be thrown away. Thus, in contrast with the above-described prior art method, the presently described method for using the sample container and filtration apparatus significantly reduces the possibility of contamination of the sample and during the sample testing procedure relative to the conventional devices and procedures, provides more consistent and reliable test results, and significantly reduces the number of preparation and testing steps for the testing procedure.

The materials for the sample container and/or base may comprise any convenient material. However, where use as a disposable is desired, inexpensive moldable materials may be preferable. For example, moldable plastics, such as polypropylene or styrene, without limitation, may be used. The sample container may also include or be packaged with materials intended to neutralize chlorinated water, such as sodium theosulfate.

The filter material may be any convenient filter. For example, for coliform, fecal or other biological water testing, standard membrane filters, as are known in the industry, may be used.

It will be apparent to those skilled in the art that, while exemplary embodiments have been shown and described, various modifications and variations can be made to the sample container and filtration apparatus disclosed herein without departing from the spirit or scope of the invention. Accordingly, it is to be understood that the various embodiments have been described by way of illustration and not limitation.

What is claimed is:

1. A sample container and filtration apparatus, comprising:
a cover;
an interior volume defined by the walls of the sample container; and
a sliding member, the sliding member configured to open a pathway between the interior volume of the sample container and an exterior.

2. A sample container and filtration apparatus in accordance with claim 1, wherein the sliding member is configured to open a pathway between the interior volume of the sample container and an exterior during mating of the sample container and a complementary base.

3. A sample container and filtration apparatus in accordance with claim 1, wherein the sliding member is configured to move into the interior volume of the sample container.

4. A sample container and filtration apparatus in accordance with claim 3, wherein the sliding member comprises a valve that includes at least one vent.

5. A sample container and filtration apparatus in accordance with claim 4, wherein the valve includes a plurality of vents around a circumference of the valve.

6. A sample container and filtration apparatus in accordance with claim 1, wherein the sliding member includes a rounded or conical upper surface to prevent standing fluid from accumulating.

7. A sample container and filtration apparatus in accordance with claim 1, wherein a lower portion of the walls of the sample container are rounded to prevent standing fluid from accumulating.

8. A sample container and filtration apparatus in accordance with claim 1, wherein the cover includes a living hinge.

9. A sample container and filtration apparatus in accordance with claim 1, wherein the cover includes a locking tab.

10. A sample container and filtration apparatus in accordance with claim 9, wherein the locking tab is configured to break during opening of the tab as a visual indication that the container has been opened.

11. A sample container and filtration apparatus in accordance with claim 1, wherein a lower external portion of the sample container including the sliding member includes a removable cap or cover that maintains a sterile field thereunder until such cap or cover is removed.

12. A sample container and filtration apparatus in accordance with claim 1, wherein a lower external portion of the sample container includes a ridge or projection configured to engage a groove or hole on a complementary base component.

13. A sample container and filtration apparatus in accordance with claim 1, wherein the sample container is disposable.

14. A sample container and filtration apparatus in accordance with claim 13, wherein at least the wall and cap portions of the sample container are plastic.

15. A sample container and filtration apparatus in accordance with claim 14, wherein at least the wall and cap portions of the sample container are a polypropylene or a styrene.

16. A fluid testing filtration method utilizing a sample container and filtration apparatus, comprising:
providing a sample container and filtration apparatus in sterile packaging, the sample container and filtration apparatus comprising:
a first cover;
an interior volume defined by the walls of the sample container; and
a broachable surface, the broachable surface configured to open a pathway between the interior volume of the sample container and an exterior;
removing a second cover over a lower external portion of the sample container, which portion includes said broachable surface;
mating said lower portion of the sample container with a vacuum filtration base, which base is configured to support a filter;
opening a portion of the sample container above the top level of the sample fluid to atmosphere;
applying a vacuum to the assembly through the base portion;
disengaging the sample container from the base; and
removing the filter for culture or analysis.

17. A method in accordance with claim 16, further comprising:
removing the sample container from sterile packaging;
collecting a sample therein;
securing the cover on the sample container; and
transporting the sample to a testing facility.

18. A method in accordance with claim 16, wherein the sample container and the base include complementary ridge in groove or projection in hole features on the mating surfaces thereof.

19. A method in accordance with claim 18, wherein the mating produces an audible or tactile indication of complete assembly.

20. A method in accordance with claim 16, wherein said first cover includes a locking tab that is configured to break upon opening the cover for indication that a sample has or has not been opened prior to testing.

21. A method in accordance with claim 16, wherein said broachable surface comprises a sliding valve that is configured to open a pathway between the interior volume of the sample container and an exterior during mating of the sample container and the base.

22. A method in accordance with claim 21, wherein the sliding member is configured to move into the interior volume of the sample container.

23. A method in accordance with claim 22, wherein the sliding member comprises a valve that includes at least one vent.

24. A method in accordance with claim 23, wherein the valve includes a plurality of vents around a circumference of the valve.
